(19) United States
(12) Patent Application Publication
(10) Pub. No.: US 2011/0182824 A1
(43) Pub. Date: Jul. 28, 2011

(54) METHOD AND DOSAGE FORM TO CONFIRM COMPLIANT USE OF A BIOACTIVE AGENT

(76) Inventors: Richard C. FUISZ, Beverly Hills, CA (US); Joseph M. Fuisz, Surfside, FL (US)

(21) Appl. No.: 12/704,742

(22) Filed: Feb. 12, 2010

Related U.S. Application Data
(63) Continuation-in-part of application No. 12/692,737, filed on Jan. 25, 2010.

(51) Int. Cl. A61K 49/00; A61K 9/00; A61P 43/00

(52) U.S. Cl. 424/9.7; 424/400

(57) ABSTRACT

A dosage form includes a dose of at least one bioactive agent and a substance that is visible to a normal light or to a special light source in the buccal, nasal, vaginal, rectal or ostomic cavity of a patient in order for medical personnel to determine if the patient is being compliant with taking medication or that a person is not truthful in denial of taking medication. The substance is either coating the at least one bioactive agent or intermixed with the at least one bioactive agent in a delivering body, and leaves behind a stain in the cavity for a predetermined period for compliance/abuse assessment.

### CROSS-REFERENCE TO RELATED APPLICATION

This application is a continuation-in-part of prior application Ser. No. 12/692,737, filed Jan. 25, 2010, the contents of which are incorporated herein by reference.

### BACKGROUND OF THE INVENTION

Misuse of bioactive agents is a significant public health issue, particularly in the case of opiates and other drug classes that are subject to abuse. Broadly speaking, such abuse may take a number of forms. The patient may not use the prescribed drug at all and instead divert the drug for illicit resale. A patient or other user may misuse the drug in a manner other than its intended use, for example by hoarding the drug and then taking a large amount in a single administration to get “high.” Alternatively, the patient may take a drug intended for oral use and snort the drug or “mainline” the drug through injection. In other instances, an individual may uses basic chemical processes to separate various components of a drug dosage form so as to misuse a selected portion thereof.

Of course, non compliance with intended therapeutic regimes is not limited to “abuse” drugs. Other reasons for lack of adherence or noncompliance are numerous, possibly including that the patient forgot to take the medication, had symptoms that went away, wanted to save money, did not believe the drugs were effective, experienced unwanted side effects, may have feared addiction, etc.

Many believe that physicians' fear of abuse potential leads to an under prescribing of certain abuse prone medications which offer great therapeutic value when properly used.

Drug companies have attempted to curb abuse in a variety of ways. One method has been to “lock” the drug in a matrix so that the drug can only be used through an intended mode of administration. One such example is Flannel's “trigger lock” technology that seeks to prevent extraction of the drug from the dosage form by crushing or other traditional separation methods (see US 2008/0008659 A1; see also EP-A-1203209). Others have suggested the manufacture of tablets with great physical strength which are difficult to crush (US 2005/0031546 A1). Another drug specific approach has been to pair an opiate with an agonist in a sublingual dosage unit wherein the agonist is not sublingually absorbed in sufficient strength to impair the intended therapeutic use of the opiate (Lewis at al U.S. Pat. No. 4,582,835; see also Farrel US 2003/0026838 A1).

In sum, one can say that these attempts are directed at preventing the user from using the drug in a way other than the intended modality by creating physical and or chemical barriers to such misuse.

### SUMMARY OF THE INVENTION

The present invention relates to a dosage form including a dose of at feast one bioactive agent and a substance that is visible to a normal light or to a special light source in the buccal, vaginal, rectal, nasal or ostomic cavity of a patient in order for medical personnel to determine if the patient is being compliant with taking medication or that a person is not truthful in denial of taking medication, wherein the substance is either coating the at least one bioactive agent or intermixed with the at least one bioactive agent in a delivering body.

### DETAILED DESCRIPTION OF THE INVENTION

The present invention is a methodology for effective monitoring if a drug is taken as directed.

The present invention also seeks to avoid abuse (and non-compliance). However, the present invention functions in a way which is distinct from, and novel with respect to, the prior art. Rather than seeking to lock the drug in a dosage form for a particular use, the present invention is a method of easily confirming compliant use of the product. Specifically, the present invention involves the use of a long acting staining agent for use with an oral dosage form such that the mucosa is stained for a period to confirm use by the patient (or failure to use, as the case may be) of the dosage form as intended. When failure to use is discovered by healthcare professionals, such failure can be appropriately dealt with.

In one aspect of the present invention, an oral medication includes a dose of at least one bioactive agent and a substance that is visible to a normal light or to a special light source in a patient's mouth in order for medical personnel to determine if the patient is being compliant with taking medication.

The present invention can take the form of a solid dosage form including a dose of at least one bioactive agent and a substance that is visible to a normal light or to a special light source in the buccal, lingual, nasal, sublingual, vaginal, rectal or ostomic cavity of a patient in order for medical personnel to determine if the patient is being compliant with taking medication, wherein the substance is either coating the at least one bioactive agent or intermixed with the at least one bioactive agent in a delivering body, such as a capsule.

The dosage form can be any solid dosage form currently existing or hereafter developed, e.g., capsule, tablet, film, sheet, quick dissolve solid, medicated lozenge, medicated lollipop, etc. The present invention may also be used with a liquid dosage form, including inter alia a solution or suspension, or such other liquid dosage form as may be developed in the future.

The bioactive agent can include, but are not limited to ace-inhibitors, antianginal drugs, anti-arrhythmias, anti-asthmatics, anti-cholesterolemics, analgesics, anesthetics, anti-convulsants, anti-depressants, anti-diabetic agents, anti-diarrhea preparations, antidotes, anti-histamines, anti-hypertensive drugs, anti-inflammatory agents, anti-lipid agents, anti-manics, anti-nauseants, anti-stroke agents, anti-thyroid preparations, anti-tumor drugs, anti-viral agents, acne drugs, alkaloids, amino acid preparations, anti-tussives, anti-uricemic drugs, anti-viral drugs, anabolic preparations, systemic and non-systemic anti-infective agents, anti-neoplastics, anti-parkinsonian agents, anti-rheumatic agents, appetite stimulants, biological response modifiers, blood modifiers, bone metabolism regulators, cardiovascular agents, central nervous system stimulates, cholinesterase inhibitors, contraceptives, decongestants, dietary supplements, dopamine receptor agonists, endometriosis management agents, enzymes, erectile dysfunction therapies, fertility agents, gastrointestinal agents, homeopathic remedies, hormones, hypercalcemia and hypocalcemia management agents, immunomodulators, immunosuppressives, migraine preparations, motion sickness treatments, muscle relaxants, obesity management agents, osteoporosis preparations, oxytocics, parasympatholytics, parasympathomimetics, prostaglandins, psychotherapeutic agents, respiratory agents, sedatives, smoking cessation aids, sympatholytics, tremor preparations, urinary tract agents, vasodilators, laxatives, antacids, ion exchange resins, anti-pyretics, appetite suppressants, expectorants, anti-anxiety agents, anti-ulcer agents, anti-inflammatory substances, coronary dilators, cerebral dilators, peripheral vasodilators, psycho-tropics, stimulants, anti-hypertensive drugs, vasoconstrictors, migraine treatments, antibiotics, tranquilizers, anti-psychotics, anti-tumor drugs, anti-coagulants, anti-thrombotic drugs, hypnotics, anti-emetics, anti-nauseants, anti-convulsants, neuromuscular drugs, hyper- and hypo-glycemic agents, thyroid and anti-thyroid preparations, diuretics, anti-spasmodics, terine relaxants, anti-obesity drugs, erythropoietic drugs, anti-asthmatics, cough suppressants, mucolytics, DNA and genetic modifying drugs, and combinations thereof. The analgesics can include opiates and opiate derivatives, such as oxycodone (available as Oxycontin®), ibuprofen, aspirin combinations, acetaminophen, and combinations thereof that may optionally include caffeine. The analgesics can include any pain medication. These also include drug pre dosage units as, for example, those volitalized and then inhaled. These mark the nasal mucosa.

The present invention is especially effective for use in connection with a bioactive agent that is adjudged by the health care provider to be especially subject to abuse by the patent or general populace, especially bioactive agents prescribed for a limited period of time. This category is mainly pain directed drugs but is not limited thereto. Such bioactive agents include, but are not limited to buprenorphine (Buprenex), butorphanol (Stadol), codeine (Hydrocodone), hydromorphone (Dilaudid, Dilaudid-5, Dilaudid-HP, Hydrostat IR), levorphanol (Levo-Dromoran), meperidine (Demerol), methadone (Dolophine, Methadose), morphine (Astramorph PF, AVINZA, Duramorph, Kadian, M S Contin, MSIR, Oramorph SR, Rescudose, Roxanol), nalbuphine (Nubain), oxycodone (OxyContin, Roxicodone), oxymorphone (Numorphan), pentazocine (Talwin), propoxyphene (Cotanal-65, Darvon), tramadol (Ultram), tramadol and acetaminophen combination (Ultracet), butalbital, acetaminophen, and caffeine combination (Femcet, Fioricet, Esgic, Esgic-Plus), butalbital, aspirin, and caffeine combination (Fiorinal), butalbital, acetaminophen, caffeine, and codeine combination (Fioricet with Codeine), hydrocodone and ibuprofen combination (Hydrostal IR, Vicoprofen), pentazocine/naloxone (Talwin NX), acetaminophen and codeine combination (Capital with Codeine, Margesic #3, Phenaphen with Codeine, Tylenol with Codeine), dihydrocodeine, acetaminophen, and caffeine combination (DHCplus), hydrocodone and acetaminophen combination (Allay, Anexsia 5/500, Anexsia 7.5/650, Dolacet, Dolagesic, Duocet, Hycomed, Hydrocet, Hydrogesic, HY-PHEN, Lorcet 10/650, Lorcet-HD, Lortab, Panacet 5/500, Panlor, Stagesic, T-Gesic, Ugesic, Vicodin, Zydone), oxycodone and acetaminophen combination (Endocet, Percocet, Roxicet, Roxilox, Tylox), pentazocine and acetaminophen combination (Talacen), propoxyphene and acetaminophen combination (Darvocet-N 50, Darvocet-N 100, E-Lor, Propacet 100), aspirin, caffeine, and dihydrocodeine combination (Synalgos-DC), aspirin and codeine combination (Empirin with Codeine), hydrocodone and aspirin combination (Damason-P, Lortab ASA, Panasal 5/500), oxycodone and aspirin combination (Endodan, Percodan, Percodan-Demi, Roxiprin), pentazocine and aspirin combination (Talwin Compound), and propoxyphene, aspirin, and caffeine combination (Darvon Compound-65, PC-Cap, Propoxyphene Compound-65)

The “staining agent” in the dosage form is visible to a normal or special light source, e.g., a black light source, etc. in order for medical personnel to determine if the patient is being compliant with taking medication. The substance can be a dye/stain (selected for being edible and also having a half life, e.g., a half buccal life, of, e.g., 6-24 hours (or shorter or longer duration)), and/or a substance that is a dye/stain, or a substance other than a dye/stain, e.g., a composition that shows under certain light. As used herein, “staining agent” is used to mean a substance, whether a dye or stain, that is visible in normal light or when exposed to a special light source. The substance can be incorporated into any oral or other dosage unit. The stain from the substance is then visible in the patient's mouth, e.g., on the patients tongue, sublingual space, buccal space or a patient's vaginal, rectal or ostomic cavity for a predetermined time period.

The basic principle is to include a medically safe and accepted staining material in the dosage form, especially (but not limited to) those dosage forms that have a residence time of 10 seconds or more in the mammalian oral, vaginal, rectal or ostomic cavity. It is possible to use this methodology with conventionally administered tablets, which have a much shorter residence time, as is successfully demonstrated in the examples below, but is easier to employ the system where the residence time is greater.

It is preferable that the staining agent be non-toxic, such that the staining agent does not cause damage in the event that the dosage form is misused by snorting or mainlining. It is an express intention of this invention that in certain embodiments, the staining agent may stain the nasal mucosa in the event that the dosage form is crushed and snorted or volatilized thereby demonstrating such misuse.

It is preferable that the staining effect of the staining agent have a controlled, limited duration. Ideally, the staining effect will correspond to the expected use of the drug. For example, an agent that is administered once daily should have a staining effect that lasts, when used as directed, approximately 24 to 30 hours.

When the mucosa is effectively stained by the present method, the presence of the stain can be confirmed in the normal course by healthcare personnel. While, admittedly a temporary stain is not considered desirable, it is contemplated that particularly for powerful abuse prone drugs, that the patient will not be bothered by such staining. For example, a patient who is prescribed the fentanyl lolly pop will presumably not be bothered by a temporary staining of the buccal cavity associated with its use. It will also give health care personal a double check that the agent has been given and taken. This adds an additional layer of safety.

Stain concentration as a percentage of the dosage form composition, for a given dye agent, will control the speed during which the staining occurs, as well as the duration of the stain on the target area.

The necessary stain concentration gradient depends on residence time for proper use of the dosage form.

It may be desirable to that the dosage form not stain immediately, but rather require the intended residence time for the product in order to stain. As a result, the stain confirms that the dosage form has been used as intended, and not inserted briefly in for example the mouth and then removed for unintended use.

It is also contemplated that two or more staining agents may be used in a single dosage form. These staining agents may be mixed equally throughout the dosage form, or separated and or segregated in different areas of the dosage form. For example, the outer portion, or but not limited to coating, of a tablet may contain one staining agent of a certain color, and the inner portion may contain a second staining agent of a different color. The effect of the use of two different staining agents may be to further confirm that the entire dosage form as been used as intended. The absence of the staining effect caused by the exposure to the two successive staining agents indicates that the dosage form as not been completed used as intended, and may have been partially diverted for potential misuse.

The stain may be placed in an outer coating on the dosage form but the preferred embodiment is throughout the dosage form for compliance reasons. Tablet coating technologies are understood in the industry. Coatings may also be used without a staining agent to prevent a staining effect from occurring on the fingers of individuals handling the dosage form.

The dye or stain may be combined with, or attached to an agent that adheres to a mucosal surface. This allows the dose to be retained on the mucosal surface, e.g., on the tongue, long enough for the dye or stain to stain the mucosal surface.

As an example, but without limitation, the stain Gentian Violet in 0.0001% to 20% concentration (measured by percentage mass of the entire dosage form) can be used as a substance that is visible to a normal light or to a special light source in the mammalian oral, vaginal, rectal, nasal or ostomic cavity in order for medical personnel to determine if the patient is being compliant with taking medication. This material is understood to be safe for all forms of delivery in the mammal (see Piatt and Bergeson, “Gentian Violet Toxicity” in Clinical Pediatrics, Vol. 31, No. 12, 756-757 (1992), addressing safety of Gentian Violet for treatment of thrush in human infants). It leaves behind a significant blue stain on the cavity in which it is placed thereby giving the health care provider ready knowledge of compliance and use as provided above.

In abuse and medical studies, the material has been safely used intravenously although we would consider this an abuse of the drug and the drug could be detected in the blood stream. It is understood that gentian violet when injected into a mammal may be visible through the skin (see Churchman and Herz, “The toxicity of Gentian Violet and its fate in the Animal Body” in J Exp Med., 18 (5): 579 (1913). Other staining agents may similarly be visible when injected.

Other examples of substances visible in mammalian oral, vaginal, rectal, nasal or ostomic cavity, e.g., having the desired staining effect, are described, without limitation below.

The colored triarylmethanes of Formula I and Formula II below are useful in the practice of the invention:

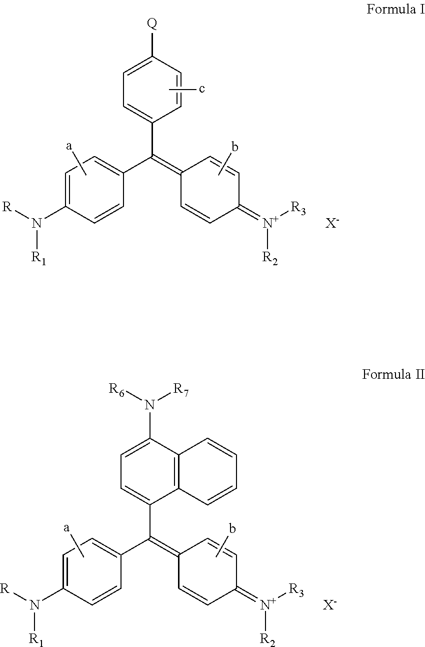

wherein R, R₁, R₂ and R₃ are independently selected from hydrogen, C₁-C₆-alkyl, substituted C₁-C₃-C₆-alkenyl, C₃-C₆-cycloalkyl, and aryl; a, b and c represent hydrogen or are independently selected from C₁-C₃-alkyl and halogen; Q is selected from hydrogen, C₁-C₆-alkyl, C₁-C₆-alkoxy, halogen and —N(R₄)R₅, wherein R₄ and R₅ are independently selected from hydrogen, C₁-C₆-alkyl, substituted C₁-C₆-alkyl, C₃-C₆-alkenyl, C₃-C₆-cycloalkyl and aryl; R₆ is selected from C₁-C₆-alkyl, C₃-C₆-cycloalkyl and aryl; R₇ is hydrogen or C₁-C₆-alkyl; X⁻ is an anion selected from Cl⁻, Br⁻, I⁻, CH₃CO₂⁻, HSO⁻₄ᵢ,

$$\frac{{ZnCl}_{4}^{=}}{2} ,$$

and the like.

Compounds of the Formulae I and II range in color from red to green. In general, compounds of the Formula I wherein Q is an —N(R₄)R₅ group are red to violet, usually violet. Compounds of the Formula I wherein Q is hydrogen, or is a group selected from hydrogen, halogen, C₁-C₆-alkyl and C₁-C₆-alkoxy, usually range in color from bluish-green to green and compounds of Formula II are usually blue.

Some of the preferred compounds are the violet compounds of Formula I, wherein Q is —N—(R₄)R₅, i.e., compounds of the following Formula III:

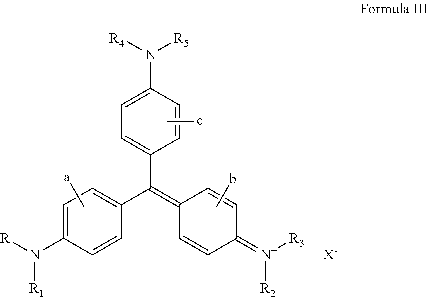

wherein R, R₁, R₂, R₃, R₄ and R₅ are independently selected from hydrogen, methyl and ethyl; a, b and c are hydrogen; X⁻ is C⁻. The most preferred compound of Formula III is the violet compound wherein R, R₁, R₂, R₃, R₄ and R₅ are methyl and X⁻ is Cl⁻, i.e., compounds of the following Formula IV:

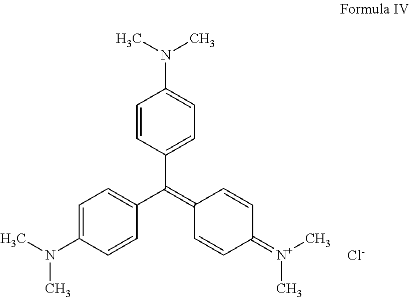

In the Colour Index International, 3^{rd} Edition (published by The Society of Dyers and Colourists with the American Association of Textile Chemists and Colorists), this compound has been assigned The Colour Index Generic Name, Basic Violet 3, and the Colour Index Constitution Number, C.I. 42555.

For the sake of clarity, it should be mentioned that the resonance structure provided above as Formula ill is only one of three possible structures, the other two having the structures in the following Formula V and VI:

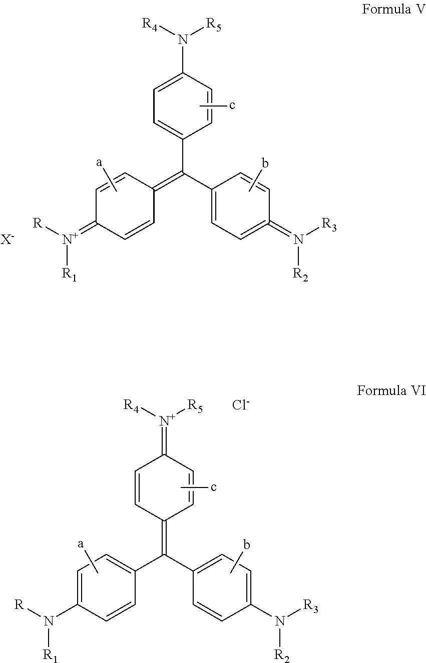

For further clarity, it should also be mentioned that two other structures have been traditionally used for compounds of the Formula III and triarylmethane type compounds in general, as the following Formula VII and Formula VIII:

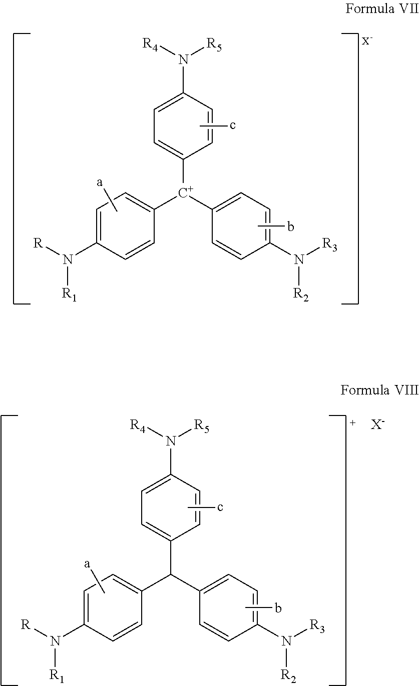

The terms “C₁-C₃-alkyl” and “C₁-C₆-alkyl” are used above to denote an aliphatic hydrocarbon radical that contains one to three and one to six carbon atoms, respectively, and the hydrocarbon radicals are either a straight chain or a branched chain.

The term “substituted C₁-C₆-alkyl” is used to denote a C₁-C₆-alkyl radical substituted with one or two groups selected from the following: halogen, hydroxy, cyano, carbonyl, aryl, C₃-C₆-cycloalkyl, succinimido, glutarimido, phthalimido, phthalimidino, 2-pyrolidino, o-benzoicsulfimido, heteroaryl, C₁-C₆-alkoxy; C₆-alkylthio, C₁-C₆-alkylsulfonyl, heteroarylthio, C₁-C₆-alkanoyloxy, aryloxy, arythio and arylsulfonyl.

The terms “C₃-C₆-alkenyl” denotes a straight or branched chain hydrocarbon radical containing three to six carbon atoms and at least one carbon-carbon double bond.

The term “C₃-C₆-cycloalkyl” denotes a saturated cycloaliphatic radical containing three to six carbon radicals.

The terms “C-₁-C₆-alkoxy”, “C₁-C₆-alkylthio”, “C₁-C₆-alkylsulfonyl” and “C₁-C₆-alkyl, alkanoyloxy” denote the following structures, respectively —O—C₁-C₆-alkyl, —SC₁-C₆-alkyl, —O₂SC₁-C₆-alkyl and —OCOC₁-C₆-alkyl.

In the terms “aryl,” “arythio,” “aryloxy” and “arylsulfonyl,” the aryl groups are selected from a phenyl group and phenyl substituted with one or two groups selected from C₁-C₆-alkyl, C₁-C₆-alkoxy, halogen and hydroxy.

In the terms “heteroaryl” and “heteroarylthio” the heteroaryl groups or heteroaryl portions of the groups are mono or bicyclo heteroaromatic radicals containing at least one hetero atom selected from the group consisting of oxygen, sulfur and nitrogen, or a combination of these, to complete the heteroaromatic ring. Examples of suitable heteroaromatic groups include: furyl, thienyl, thiazolyl, benzothiazolyl, isothiazolyl, pyrazolyl, pyrolyl, pyrimidinyl and triazolyl and such groups substituted with one or two groups selected from C₁-C₆-alkyl, C₁-C₆-alkoxy, C₃-C₆-cycloalkyl, C₁-C₆-alkylthio, aryl, arylthio, aryloxy and halogen.

The term “halogen” is used to include fluorine, chlorine, bromine and iodine.

The term “carbamyl” is used to represent the group having the formula —CON(R₈R₉), wherein R₈ and R₉ are independently selected from hydrogen, C₁-C₆-alkyl and aryl.

Triarylmethane colorants of Formula I and Formula II are generally well-known and many are listed in the Colour Index International, 3^{rd}. Ed., Vol. 4, pages 4380-4407. Many references are also provided there to synthetic methods for preparing the compounds. Also, provided by the Colour Index International, 3^{rd} Ed., Vol. 5 are Colour Index Generic Names (C.I. Generic Names) and Colour Index Constitution Numbers (CA. Constitution Numbers) for many specific structures. Colour Index International, 3^{rd}. Ed., Vol. 5, also provides a list of manufacturers/suppliers for each C.I. Generic Name.

Examples 1-15 in Table I below provide several specific compounds included in Formula I or Formula II above, which are useful in the practice of the present invention. Also, the C.I. Generic Names and C.I. Constitution Numbers are provided for the colored compounds of Examples 1-15.

TABLE 1
Typical Useful Triphenylmethane Dye

| Example Number | Structure | C.I. Generic Name | C.I. Comparison Number |
|---|---|---|---|
| 1 | Formula IV | Basic Violet 3 | C.I. 42555 |
| 2 | Formula IX below | Basic Violet 4 | C.I. 42600 |
| 3 | Formula X below | Basic Violet 2 | C.I. 42520 |
| 4 | Formula XI below | Basic Violet 1 | C.I. 42535 |
| 5 | Formula XII below | Basic Violet 14 | C.I. 42510 |
| 6 | Formula XIII below | Basic Red 9 | C.I. 42500 |
| 7 | Formula XIV below | Basic Violet 23 | C.I. 42557 |
| 8 | Formula XV below | Basic Green 1 | C.I. 42040 |
| 9 | Formula XVI below | Basic Green 4 | C.I. 42000 |
| 10 | Formula XVII below | Basic Blue 1 | C.I. 42025 |
| 11 | Formula XVIII below | Basic Blue 8 | C.I. 42563 |
| 12 | Formula XIX below | Basic Blue 11 | C.I. 44040 |
| 13 | Formula XX below | Basic Blue 7 | C.I. 42595 |
| 14 | Formula XXI below | Basic Blue 26 | C.I. 44045 |
| 15 | Formula XXII below | Basic Blue 15 | C.I. 44085 |

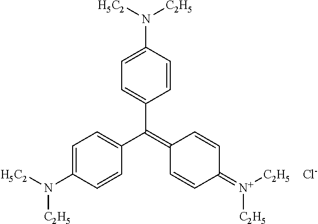

Formula IX 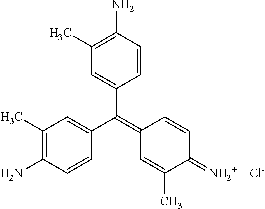
Formula X 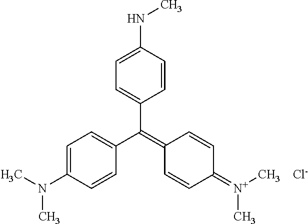
Formula XI 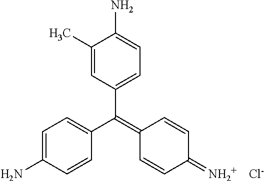
Formula XII 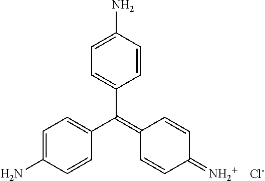
Formula XIII 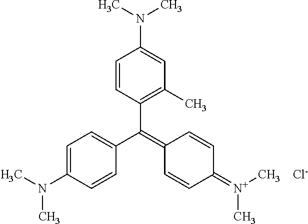
Formula XIV 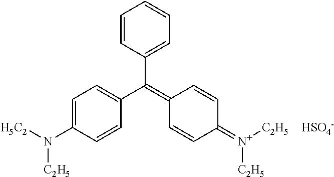
Formula XV 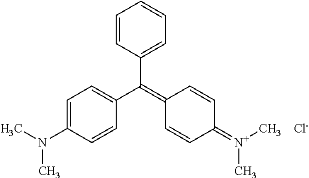
Formula XVI 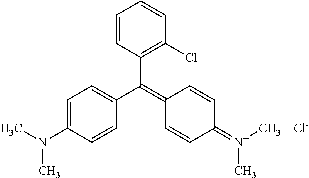
Formula XVII 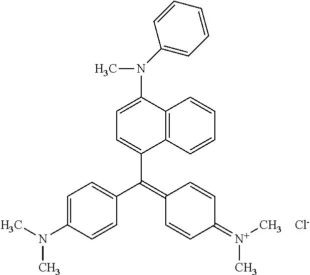
Formula XVIII 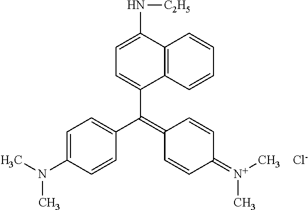
Formula XIX 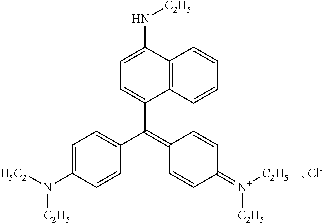
Formula XX 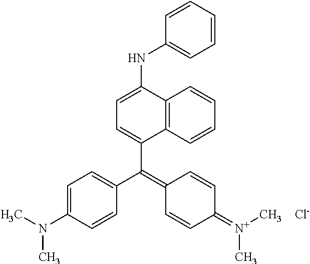
Formula XXI 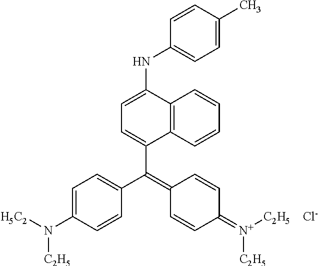
Formula XXII

There follow some examples. In the following examples, Gentian Violet Powder HARLECO Certified Biological Stain was acquired from Gallade Chemical.

### EXAMPLE A

9.76 grams of Domino's confectionary sugar were placed in a crucible together with 0.24 grams of Gentian Violet powder. This blend was mixed thoroughly to create a 2.4% Gentian Violet concentration “master batch”.

Four TUMS® Ultra Strength 1000 tablets were pulverized using a mortar and pestle. Five drops of water were added to the pulverized TUMS® tablets and the blend was mixed thoroughly together.

9.6 grams of the crushed, wetted TUMS® tablets as described above were combined with 0.4 gram of the 2.4% Gentian Violet “master batch.” This blend was mixed thoroughly to make concentration of 0.096% Gentian Violet. 0.5 grams of this mix was placed into a single tablet press to make tablet. Eight more such tablets were made in the same fashion.

### EXAMPLE B

The method of Example A was repeated with the use of a lower concentration of 2.4% Gentian Violet “master batch” to make eight tablets with a final concentration of 0.048% Gentian Violet.

### EXAMPLE C

A healthy male volunteer placed the tablet of Example A in the buccal cavity and allowed the tablet to dissolve in his buccal cavity over a period of twenty five minutes. A blue stain resulted in the buccal cavity. This blue buccal stain was not visible when the subject conversed or ate. The stain lasted for approximately thirty hours, and disappeared without any permanent effect.

### EXAMPLE D

A healthy male volunteer placed the tablet of Example B in the buccal cavity and allowed the tablet to dissolve in his buccal cavity over a period of twenty five minutes. A blue stain resulted in the buccal cavity. This blue buccal stain was not visible when the subject conversed or ate. The stain lasted for approximately six hours, and disappeared without any permanent effect.

### EXAMPLE E

Four TUMS® Ultra Strength 1000 were crushed using mortar and pestle. Five drops of water where added to the crushed TUMS® tablets and they were mixed thoroughly. Then 0.1 gram of Gentian Violet was added to 1.9 gram wetted crushed TUMS®. The composition was mixed again, and then tablets were pressed (approximate total mass of 650 mg for each tablet) using a single tablet press, resulting in tablet with a concentration of 5%.

### EXAMPLE F

A healthy male volunteer placed the a tablet made in Example E on his tongue and swallowed the tablet after approximately one second, using the same procedure that one typically uses for swallowing a conventional tablet. This administration resulted in a stain on the tongue that lasted over twelve hours despite the taking of routine meals and snacks as well as a tooth brushing.

### EXAMPLE G

Using the same method as in Example E, tablets containing 10% Gentian Violet were made. A health male volunteer took one such tablet using the same procedure as Example F. A stain was visible on his tongue in normal light for over twenty seven hours, despite the taking of routine meals, snacks and tooth brushing.

### EXAMPLE H

A healthy male volunteer took a tablet made in accordance with Example A and crushed the tablet. He then “snorted” the resulting powder. The result was to stain the nasal mucosa of the volunteer.

What is claimed is:

1. An oral medication comprising a dose of at least one bioactive agent and a substance that leaves a stain or mark in a patient's mouth visible in normal light or when exposed to a special light source in order for medical personnel to determine if the patient is being compliant with taking medication.

2. The oral medication according to claim 1, wherein the substance is visible for a predetermined time period in the patient's mouth.

3. The oral medication according to claim 1, wherein the substance is Gentian Violet.

4. The oral medication according to claim 1, wherein the substance is a colored triarylmethanes of the following Formula I or Formula II: 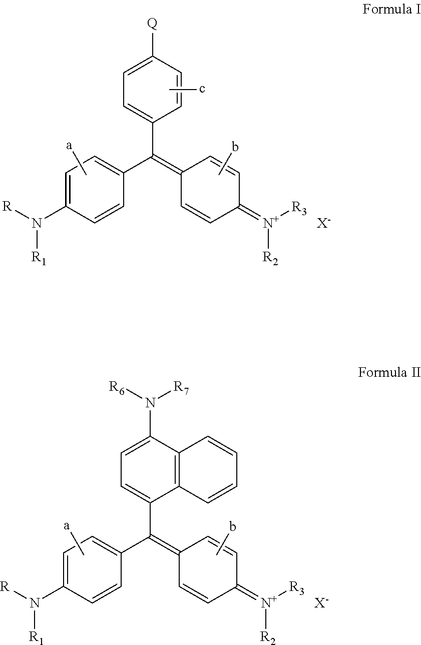
wherein R, R₁, R₂ and R₃ are independently selected from the group consisting of hydrogen, C₁-C₆-alkyl, substituted C₁-C_{6—}alkyl, C₃-C₆-alkenyl, C₃-C₆-cycloalkyl, and aryl; a, b and c represent hydrogen or are independently selected from the group consisting of C₁-C₃-alkyl and halogen; Q is selected from the group consisting of hydrogen, C₁-C₆-alkyl, C₁-C₆-alkoxy, halogen and —N(R₄)R₅, wherein R₄ and R₅ are independently selected from the group consisting of hydrogen, C₁-C₆-alkyl, substituted C₁-C₆-alkyl, C₃-C₆-alkenyl, C₃-C₆-cycloalkyl and aryl; R₆ is selected from the group consisting of C₁-C₆-alkyl, C₃-C₆-cycloalkyl and aryl; R₇ is hydrogen or C₁-C₆-alkyl; and X⁻ is an anion.

5. The oral medication according to claim 4, wherein the anion is selected from the group consisting of Cl⁻, Br⁻, I⁻, CH₃CO₂⁻, HSO⁻₄ᵢ,
$$\frac{{ZnCl}_{4}^{=}}{2} .$$

6. The oral medication according to claim 1, wherein the dose is in liquid form.

7. The oral medication according to claim 1, wherein the dose is in solid form.

8. The oral medication according to claim 7, further comprising an agent that adheres the dose to a surface of the buccal cavity for a period of time.

9. A dosage form comprising a dose of at least one bioactive agent and a substance that is visible to a normal light or to a special light source in the buccal, vaginal, nasal, rectal or ostomic cavity of a patient in order for medical personnel to determine if the patient is being compliant with taking medication, wherein the substance is either coating the at least one bioactive agent or intermixed with the at least one bioactive agent in a delivering body.

10. The dosage form according to claim 9, wherein the substance is coated in an outer surface of the at least one bioactive agent.

11. The dosage form according to claim 9, wherein the substance is intermixed with the at least one bioactive agent in the delivering body.

12. The dosage form according to claim 9, wherein the delivering body is a capsule.

13. The dosage form according to claim 9, wherein the substance is Gentian Violet.

14. The oral medication according to claim 6, wherein the substance is a colored triarylmethanes of the following Formula I or Formula II: 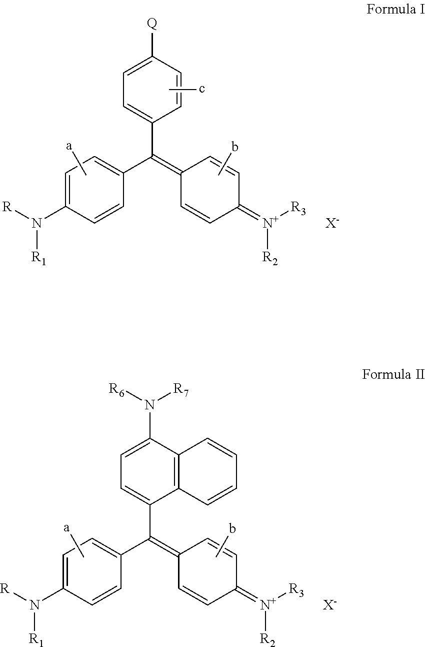
wherein R, R₁, R₂ and R₃ are independently selected from the group consisting of hydrogen, C₁-C₆-alkyl, substituted C₁-C_{6—}alkyl, C₃-C₆-alkenyl, C₃-C₆-cycloalkyl, and aryl; a, b and c represent hydrogen or are independently selected from the group consisting of C₁-C₃-alkyl and halogen; Q is selected from the group consisting of hydrogen, C₁-C₆-alkyl, C₁-C₆-alkoxy, halogen and —N(R₄)R₅, wherein R₄ and R₅ are independently selected from the group consisting of hydrogen, C₁-C₆-alkyl, substituted C₁-C₆-alkyl, C₃-C₆-alkenyl, C₃-C₆-cycloalkyl and aryl; R₆ is selected from the group consisting of C₁-C₆-alkyl, C₃-C₆-cycloalkyl and aryl; R₇ is hydrogen or C₁-C₆-alkyl; and X⁻ is an anion.

15. The dosage form according to claim 14, wherein the anion is selected from the group consisting of Cl⁻, Br⁻, I⁻, CH₃CO₂⁻, HSO⁻₄ᵢ,
$$\frac{{ZnCl}_{4}^{=}}{2} .$$

16. The dosage form according to claim 9, wherein the delivering body is in solid form.

17. The oral medication according to claim 16, further comprising an agent that adheres the delivering body to a surface of the buccal cavity for a period of time.
